(19) United States
(12) Patent Application Publication
(10) Pub. No.: US 2022/0380488 A1
(43) Pub. Date: Dec. 1, 2022

(54) ULTRA-LOW MOLECULAR WEIGHT HYALURONIC ACID AND PREPARATION METHOD THEREFOR

(71) Applicant: NANJING HANXIN PHARMACEUTICAL TECHNOLOGY CO. LTD, Nanjing (CN)

(72) Inventors: Yonggang XU, Nanjing (CN); Chuangen TANG, Nanjing (CN); Song CHEN, Nanjing (CN); Haoning ZHANG, Nanjing (CN)

(21) Appl. No.: 17/845,248

(22) Filed: Jun. 21, 2022

Related U.S. Application Data
(63) Continuation of application No. PCT/CN2020/091956, filed on May 23, 2020.

(30) Foreign Application Priority Data
Dec. 21, 2019 (CN) 201911331251.7

(51) Int. Cl. C08B 37/08; A61P 17/02; A61K 8/73; A61Q 19/00

(52) U.S. Cl. CPC C08B 37/0072; A61P 17/02; A61K 8/735; A61Q 19/00

(57) ABSTRACT

A macromolecular hyaluronic acid is used as a raw material, and is subjected to production processes such as hyaluronidase hydrolysis, heating and inactivation, activated carbon filtration, and spray and dry to obtain an ultra-low molecular weight hyaluronic acid product having an average molecular weight of less than 1200 Da. The product is a mixture of hyaluronic acid disaccharide to dodecaose. The content of hyaluronic acid disaccharide is 5-40%. The content of hyaluronic acid tetrasaccharide is 40-70%. The content of hyaluronic acid hexasaccharide is 10-30%. The content of hyaluronic acid octasaccharide is 1-15%. The content of hyaluronic acid decaose is 1-10%. The content of that higher than hyaluronic acid decaose is less than 6%. Compared with other available low molecular hyaluronic acid, the product has a more significant permeation, moisturizing, and repair promotion capability, and can be widely used in fields of medical products, health care products, cosmetics and the like.

### CROSS-REFERENCE TO RELATED APPLICATIONS

This application is a continue application of international application of PCT application No. PCT/CN2020/091956, filed on May 23, 2020, which claims the priority benefit of China application No. 201911331251.7, filed on Dec. 21, 2019. Each of the above-mentioned patent applications is hereby in its entirety incorporated by reference herein and made a part of this specification.

### TECHNICAL FIELD

The present disclosure relates to the field of biochemical technology, particularly to an ultra-low molecular weight hyaluronic acid and a preparation method therefor.

### BACKGROUND

Hyaluronic acid (HA, that is, macromolecular hyaluronic acid that is also called as hyaluronic acid) is an acidic straight-chain poly-mucopolysaccharide which is formed by repeated arrangement of (1-3)-2-N-acetamino-2-deoxy-D-glucose-O-ß-D-glucuronic acid disaccharide, which was first extracted from the vitreous body of bull's eye by Mey-cho et al in 1934, and has strong hydrophilicity and excellent moisturizing properties, and is the substance with the best moisturizing properties found in nature at present, and is recognized as the most ideal natural moisturizing factor by the international cosmetics industry. At the same time, since HA has no immunogenicity and toxicity, it is widely used in cosmetics, food and medicine industries.

According to literature studies, the molecular weight has a great influence on the biological activity of HA, and HA with different molecular weight ranges exhibits completely different physiological functions. Thanks to various functions such as good viscoelasticity, moisturizing, inhibiting inflammatory response, lubricating, HA with high molecular weight (Mr>1×10⁶) can be used in high-end cosmetic industry, ophthalmic surgery viscoelastic agent and intra-articular injection treatment. Thanks to good moisturizing, lubricating and drug release properties, HA with medium molecular weight (Mr between 1×10⁵ and 1×10⁶) is widely used in cosmetics, eye drops, skin burn healing and postoperative anti-adhesion. HA with low molecular weight (Mr is lower than 1×10⁴) and hyaluronic acid oligosaccharide exhibits extremely strong biological activity, which has functions of promoting wound healing, promoting bone and blood vessel regeneration, immune regulation, etc., and easily penetrate into the dermis. Therefore, low molecular weight hyaluronic acid has broad application prospects in the fields of food health care, cosmetics and clinical medicine.

At present, there are three main methods for preparing low molecular weight hyaluronic acid, namely, a physical method, a chemical method and a biological enzymatic method. The physical method mainly includes various manners such as heating, mechanical shearing, ultraviolet, ultrasound, radiation to promote the degradation of macromolecular HA. Although the physical treatment process is simple, and the product is easy to recycle, the product has poor stability, uneven molecular weight distribution, and low efficiency. The chemical method mainly includes hydrolysis and oxidation, hydrolysis is divided into alkali hydrolysis and acid hydrolysis, and sodium hypochlorite and hydrogen peroxide are commonly used in oxidative degradation. The chemical degradation of macromolecular HA is widely applied and the conditions are relatively mature, however, due to the complex degradation conditions of different chemical reagents, the product properties are easily affected and the purification of the product is difficult, and there is a problem of difficult disposal of waste liquid. Biological enzymatic method is a new method for degrading macromolecular HA in recent years, in which the low molecular weight hyaluronic acid is prepared through the hydrolysis of macromolecular HA by hyaluronidase. The biological enzymatic method has the advantages such as mild conditions, simple operation and high efficiency, which is the the development trend at present.

A method for efficiently separating and preparing a single-molecular-weight hyaluronic acid oligosaccharide is disclosed in Patent CN106399428B, in which a low-molecular-weight hyaluronic acid mixture is prepared by using hyaluronidase to hydrolyze macromolecular hyaluronic acid, the prepared low-molecular-weight hyaluronic acid mixture is a mixture of hyaluronic acid tetrasaccharide (HA4) to hyaluronic acid tetradecose (HA14) that is used for further separation and purification, but the proportion of oligosaccharide, average molecular weight or application are not reported. The low-molecular-weight hyaluronic acid in the present disclosure is a mixture of hyaluronic acid disaccharide (HA2) to hyaluronic acid dodecaose (HA12), the proportion of oligosaccharide is controlled by experimental conditions, the molecular weight range is narrower, and the permeability and repair effect of skin are verified by animal and active cell experiments.

A method for preparing a hyaluronic acid with a specific molecular weight is disclosed in Patent CN104178539B, in which a hyaluronic acid with an average molecular weight of 4000 Da to 370000 Da is prepared by using hyaluronidase to hydrolyze macromolecular hyaluronic acid, in which neither the proportion of oligosaccharide components in the low-molecular-weight hyaluronic acid prepared by the method, nor the application of the low-molecular-weight hyaluronic acid prepared by the method is disclosed, and the average molecular weight is 4000 Da and above. The low-molecular-weight hyaluronic acid in the present disclosure is a mixture of HA2 to HA12, the proportion of oligosaccharide is controlled by experimental conditions, the molecular weight range is narrower, and the skin permeability and repairing effects are verified by animal and active cell experiments.

A process for preparing a low-molecular-weight sodium hyaluronate is disclosed in Patent CN108484796A, in which a macromolecular hyaluronic acid is degraded into a low-molecular-weight hyaluronic acid through the degradation function of a strong oxidant, in which not the composition of the product but the permeability of the product is disclosed, in which the molecular weight range of the prepared low-molecular-weight sodium hyaluronate is 5 kDa-20 kDa, and macromolecular hyaluronic acid is degraded in a high concentration alcohol solution by using peroxide as an oxidant, in which the reaction conditions are harsh and organic solvents are used, the treatment cost of the waste liquid is high and the environmental pressure is high. The low-molecular-weight hyaluronic acid in the present disclosure is a mixture of HA2 to HA12, the proportion of oligosaccharide is controlled by experimental conditions, the molecular weight range is narrower, and the enzyme catalysis is carried out in a purified water system with mild conditions and green environmental protection.

### SUMMARY

The objectives of the present disclosure are to overcome the above-mentioned shortcomings in the prior art, and to provide a new ultra-low molecular weight hyaluronic acid and a preparation method therefor.

In order to achieve the above objectives, one of the objectives of the present disclosure is to provide the following technical solution of an ultra-low molecular weight hyaluronic acid, where an average molecular weight of the ultra-low molecular weight hyaluronic acid is less than 1200 Da, a distribution range of the molecular weight is narrow, the ultra-low molecular weight hyaluronic acid is a mixture of hyaluronic acid disaccharide to dodecaose; the content of hyaluronic acid disaccharide is 5-40%, the content of hyaluronic acid tetrasaccharide is 40-70%, the content of hyaluronic acid hexasaccharide is 10-30%, the content of hyaluronic acid octasaccharide is 1-15%, the content of hyaluronic acid decaose is 1-10%, the content of that higher than hyaluronic acid decaose is less than 6%; the structural general formula of the ultra-low molecular weight hyaluronic acid is as shown in the following formula I:

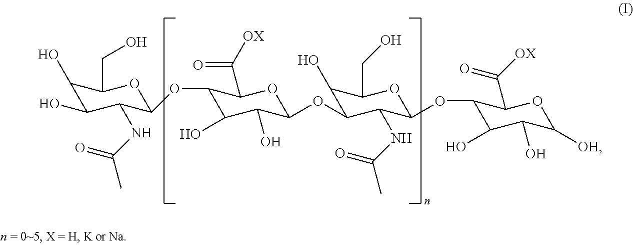

Furthermore, the average molecular weight of the ultra-low molecular weight hyaluronic acid is 500-1200 Da, and further preferably 800-1000 Da.

Moreover, the ultra-low molecular weight hyaluronic acid is a mixture of hyaluronic acid disaccharide to dodecose, the content of hyaluronic acid disaccharide is 5-40%, preferably 5-10%, the content of hyaluronic acid tetrasaccharide is 40-70%, preferably 50-70%, the content of hyaluronic acid hexasaccharide is 10-30%, preferably 20-30%, the content of hyaluronic acid octasaccharide is 1-15%, preferably 5-10%, the content of hyaluronic acid decaose is 1-10%, preferably 1-5%, the content of that higher than hyaluronic acid decaose is less than 6%, preferably less than 3%.

The second objective of the present disclosure is to provide the following technical solution of a method for preparing the ultra-low molecular weight hyaluronic acid. The method is specifically as follows: the ultra-low molecular weight hyaluronic acid with an average molecular weight of less than 1200 Daltons is obtained by enzymatically hydrolyzing the macromolecular hyaluronic acid raw material with hyaluronidase. a distribution range of the molecular weight is narrow and the ultra-low molecular weight hyaluronic acid is a mixture of hyaluronic acid disaccharide to dodecaose, Where, the content of hyaluronic acid disaccharide is 5-40%, the content of hyaluronic acid tetrasaccharide is 40-70%, the content of hyaluronic acid hexasaccharide is 10-30%, the content of hyaluronic acid octasaccharide is 1-15%, the content of hyaluronic acid decaose is 1-10%, the content of that higher than hyaluronic acid decaose is less than 6%; the molecular weight of the macromolecular hyaluronic acid is equal to or greater than 1×10⁴ Da; the structural general formula of the ultra-low molecular weight hyaluronic acid is as shown in the following formula I:

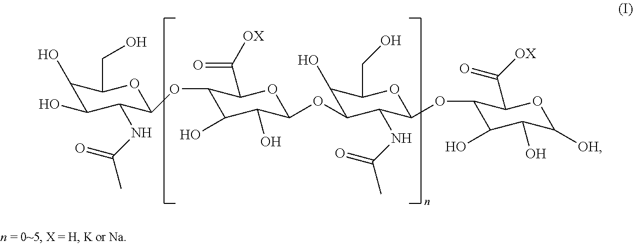

Additionally, the average molecular weight of the low molecular weight hyaluronic acid is 500-1200 Da, more preferably 800-1000 Da. The technical method involved is to utilize commercially available common macromolecular hyaluronic acid as raw material for production. The molecular weight of the macromolecular hyaluronic acid is equal to or greater than 1×10⁵ Da, more preferably 800 KDa-1600 KDa.

In addition, the ultra-low molecular weight hyaluronic acid is a mixture of hyaluronic acid disaccharide to dodecaose, the content of hyaluronic acid disaccharide is 5-10%, the content of hyaluronic acid tetrasaccharide is 50-70%, the content of hyaluronic acid hexasaccharide is 20-30%, the content of hyaluronic acid octasaccharide is 5-10%, the content of hyaluronic acid decaose is 1-5%, the content of that higher than hyaluronic acid decaose is less than 3%.

Furthermore, the hyaluronidase is a leech hyaluronidase, which is obtained by optimized expression of yeast.

Moreover, the operating conditions of the enzymatic hydrolysis reaction are as follows: the addition amount of the hyaluronidase is 1×10⁴-1×10⁵ U/mL relative to the reaction solution, more preferably 4×10⁴˜1×10⁵ U/mL; the concentration of the macromolecular hyaluronic acid raw material is 40-200 g/L, more preferably 110˜200 g/L; the reaction solvent is purified water, the enzymolysis time is 12-36 hours, more preferably 12˜24 hours; the enzymolysis temperature is 35-45° C., more preferably 35˜40° C.; the stirring speed is 100-700 rpm, more preferably 100˜400 rpm and the enzymolysis pH is 4.0-6.0.

Additionally, the reaction solution after enzymolysis reaction is heated to 80-90° C., kept for 30-60 minutes for inactivation, cooled the temperature to below 50° C., adsorbing by adding activated carbon, the reaction solution was collected and filtration.

In addition, the reaction solution is filtered and sterilized by a 0.22 μm capsule filter element for a spray-drying.

Furthermore, the obtained ultra-low molecular weight hyaluronic acid has better skin penetration and promoting repairing properties of damaged skin comparing with ordinary low-molecular-weight hyaluronic acid (3 KDa).

Moreover, the ultra-low molecular weight hyaluronic acid has applications in the fields of preparing medicines, cosmetics and health care products.

Compared with the prior art, the present disclosure has the following advantages.

1. The ultra-low molecular weight hyaluronic acid oligosaccharide mixture with an average molecular weight of less than 1200 Daltons, especially the ultra-low molecular weight hyaluronic acid mixture with an average molecular weight of 800-1000 Da, can be stably obtained by hydrolyzing the leech-type hyaluronic acid obtained by the optimized expression of yeast, and the distribution range of the molecular weight is narrow.

2. The production cycle is short, the efficiency is high, and it is suitable for industrial scale-up.

3. The product quality is stable, the ultra-low molecular weight hyaluronic acid includes a mixture of hyaluronic acid disaccharide to dodecaose; the content of hyaluronic acid disaccharide is 5-40%, the content of hyaluronic acid tetrasaccharide is 40-70%, the content of hyaluronic acid hexasaccharide is 10-30%, the content of hyaluronic acid octasaccharide is 1-15%, the content of hyaluronic acid decaose is 1-10%, the content of that higher than hyaluronic acid decaose is less than 6%.

4. Compared with the commercially available 3 KDa molecular weight products, the ultra-low molecular weight hyaluronic acid oligosaccharide mixture according to the present disclosure has a better effect of promoting penetration and hydration at the concentration of 5 mg/mL, and has a more obvious effect of repair promotion on human immortalized epidermal (HaCaT) cells damaged by hydrogen peroxide.

5. Compared with the 954 Da hyaluronic acid oligosaccharide obtained by pure chemical cleavage or chemically combined with bovine testis-type hyaluronidase enzymatic hydrolysis, the ultra-low molecular weight hyaluronic acid oligosaccharide mixture according to the present disclosure has more obvious pro-proliferation and pro-migration effects on fibroblasts at a concentration of 2.5 mg/mL.

6. Compared with the 954 Da hyaluronic acid oligosaccharide obtained by pure chemical cracking or chemically combined with bovine testis-type hyaluronidase enzymatic hydrolysis, the ultra-low molecular weight hyaluronic acid oligosaccharide according to the present disclosure has a higher healing rate for mouse skin damage, and can significantly promote the healing of skin wounds.

### BRIEF DESCRIPTION OF THE DRAWINGS

FIG. 1 is a distribution spectrum of an ultra-low molecular weight hyaluronic acid oligosaccharide prepared according to Example 1.

FIG. 2 is a distribution spectrum of an ultra-low molecular weight hyaluronic acid oligosaccharide prepared according to Example 2.

FIG. 3 is a distribution spectrum of an ultra-low molecular weight hyaluronic acid oligosaccharide prepared according to Example 4.

FIG. 4 is a HRMS mass spectrum of an ultra-low molecular weight hyaluronic acid oligosaccharide composition.

FIG. 5 is a mass spectrum of hyaluronic acid disaccharide.

FIG. 6 is a mass spectrum of hyaluronic acid tetrasaccharide.

FIG. 7 is a mass spectrum of hyaluronic acid hexasaccharide.

FIG. 8 is a mass spectrum of hyaluronic acid octasaccharide and decaose.

FIG. 9 is an infrared spectrum of the ultra-low molecular weight hyaluronic acid oligosaccharide composition.

FIG. 10 is an infrared spectrum of macromolecular hyaluronic acid.

FIG. 11 is a graph showing the results of a permeation and moisturizing detection of the ultra-low molecular weight hyaluronic acid oligosaccharide mixture.

FIG. 12 is a graph showing the detection results of the repairing effect of the ultra-low molecular weight hyaluronic acid on HaCaT cells.

FIG. 13 is a graph showing the detection results of the proliferative effect of the ultra-low molecular weight hyaluronic acid on fibroblasts.

FIG. 14 is a graph showing the detection results of the migration effect of the ultra-low molecular weight hyaluronic acid on fibroblasts.

FIG. 15 is a graph showing the detection results of the healing effect of the ultra-low molecular weight hyaluronic acid on wounded skin tissue in mice.

### DETAILED DESCRIPTION OF THE EMBODIMENTS

In order to facilitate the understanding of the present disclosure, the present disclosure will be described in detail in connection with the related drawings. The preferred embodiments of the present disclosure are shown in the accompanying drawings. However, the present disclosure can be implemented in different forms, and not limited to the embodiments described herein. In contrast, the object to provide these embodiments is to make the content of the present disclosure to be more thorough.

The materials, reagents, etc. used in the following examples can be obtained from commercial sources unless otherwise specified. The hyaluronidase is derived from Pichia pastoris GS115 used as the expression host, and the hyaluronidase gene from leech (Gen Bank accession number KJ026763) is integrated. For details, please refer to “Zhang Na” et al., Staged temperature control improves the expression of hyaluronidase in Pichia pastoris, Journal of Food and Biotechnology, 2016 (12)”, the entire contents of which can be directly incorporated into the present disclosure.

The formula for calculating the average molecular weight of the ultra-low molecular weight hyaluronic acid is shown in the following formula II:

$$Formula II : average molecular weight = \frac{{r}_{u 1} \times {M}_{W 1} + {r}_{u 2} \times {M}_{W 2} + {r}_{u 3} \times {M}_{W 3} + {r}_{u 4} \times {M}_{W 4} + {r}_{u 5} \times {M}_{W 5} + {r}_{u 6} \times {M}_{W 6}}{{r}_{t}} . \\ wherein , \underline{{r}_{t}} = {r}_{u 1} + {r}_{u 2} + {r}_{u 3} + {r}_{u 4} + {r}_{u 5} + {r}_{u 6} .$$

Where rᵤ₁ is the peak response value of component 1 (hyaluronic acid dodecaose) in the sample solution; Mwi is the molecular weight of component 1 in the sample solution;

rᵤ₂ is the peak response value of component 2 (hyaluronic acid decaose) in the sample solution; M_{W2} is the molecular weight of component 2 in the sample solution;
rᵤ₃ is the peak response value of component 3 (hyaluronic acid octasaccharide) in the sample solution; M_{W3} is the molecular weight of component 3 in the sample solution;
rᵤ₄ is the peak response value of component 4 (hyaluronic acid hexasaccharide) in the sample solution; M_{W4} is the molecular weight of component 4 in the sample solution;
rᵤ₅ is the peak response value of component 5 (hyaluronic acid tetrasaccharide) in the sample solution; M_{W5} is the molecular weight of component 5 in the sample solution;
rᵤ₆ is the peak response value of component 6 (hyaluronic acid disaccharide) in the sample solution; M_{W6} is the molecular weight of component 6 in the sample solution;
rᵤ₆ is the sum of the peak response values of component 1, component 2, component 3, component 4, component 5 and component 6 in the sample solution.

### Example 1 of Enzymatic Hydrolysis Reaction

3 L of purified water is added to a 5 L glass beaker, the stirring speed is controlled to 400 rpm, the temperature is controlled to 40° C., 1.5×10⁸ U of hyaluronidase are added, the enzyme activity of the system is 5×10⁴ U/mL, and 330 g of macromolecular hyaluronan with a molecular weight of 800 KDa are added, after all of the substance is completely dissolved, the pH of the solution is adjusted to 5.5, and the system is kept at 40° C. and stirred for 24 h.

### Example 2 of Enzymatic Hydrolysis Reaction

3 L of purified water is added to a 5 L glass beaker, the stirring speed is controlled to 100 rpm, the temperature is controlled to 35° C., 1.2×10⁸ U of hyaluronidase are added, the enzyme activity of the system is 4×10⁴ U/mL, and 330 g of macromolecular hyaluronan with a molecular weight of 800 KDa are added, after all the substance is completely dissolved, the pH of the solution is adjusted to 6.0, and the system is kept at 35° C. and stirred for 24 h.

### Example 3 of Enzymatic Hydrolysis Reaction

3 L of purified water is added to a 5 L glass beaker, the stirring speed is controlled to 400 rpm, the temperature is controlled to 40° C., 3×10⁸ U of hyaluronidase are added, the enzyme activity of the system is 1×10⁵ U/mL, and 600 g of macromolecular hyaluronan with a molecular weight of 800 KDa are added, after all the substance is completely dissolved, the pH of the solution is adjusted to 4.0, and the system is kept at 45° C. and stirred for 12 h.

### Example 4 of Activated Carbon Adsorption

3 L of the hydrolyzed solution after the reaction in Example 1 is taken, the hydrolyzed solution is heated to 80° C. and stirred for 1 h, then it is cooled to 40° C., 15 g of activated carbon are added, stirred for 30 min, and the filtrate solution is collected by filtration.

### Example 5 of Activated Carbon Adsorption

3 L of the hydrolyzed solution after the reaction in Example 4 is taken, the hydrolyzed solution is heated to 90° C. and stirred for 0.5 h, then it is cooled to 40° C., 30 g of activated carbon are added, stirred for 30 min, and the filtrate solution is collected by filtration.

### Example 6 of Spray Drying

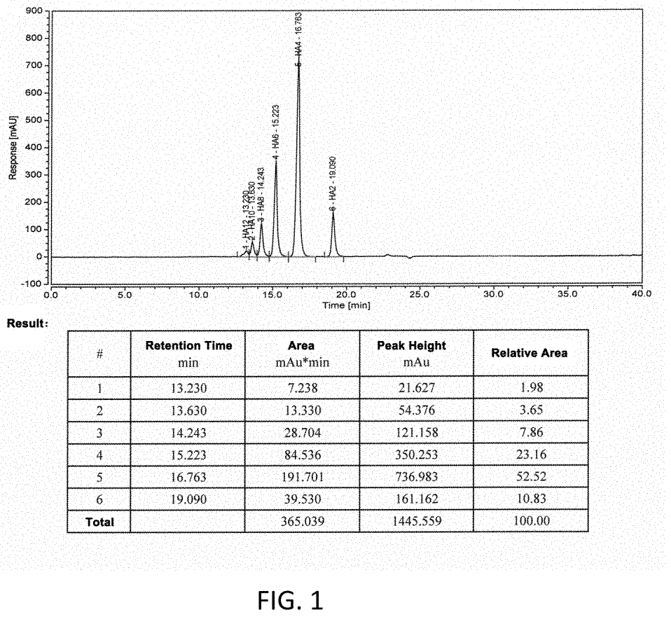

3 L of the filtrate solution obtained in Example 5 is filtered and sterilized by a 0.22 um capsule filter element, such as a capsule filter, and then spray-dried. The spray-drying parameters are that: the inlet air temperature is 120° C., the outlet air temperature is 60° C., and the flow rate is 100 rpm. 264 g of low-molecular-weight hyaluronic acid products are obtained, and the yield is 80% (that is, the proportion of 264 g of low-molecular-weight hyaluronic acid to 330 g of macromolecular hyaluronic acid raw materials). The molecular weight distribution measured by molecular exclusion chromatography is as illustrated in FIG. 1, the first component with a peak time of 13.230 min is dodecaose with a content of 1.98%; the second component with a peak time of 13.630 min is decaose with a content of 3.65%; the third component with a peak time of 14.243 min is octasaccharide with a content of 7.86%; the fourth component with a peak time of 15.223 min is hexasaccharide with a content of 23.16%; the fifth component with a peak time of 16.763 min is tetrasaccharide with a content of 52.52%; the sixth component with a peak time of 19.090 min is a disaccharide with a content of 10.83%, and therefore, the sum of the content of the mixture of hyaluronic acid disaccharide to dodecaose is 100%. The average molecular weight of low molecular weight hyaluronic acid is 954 Da, and the specific calculation process is as follows:

$$average molecular weight = \frac{7.238 \times 2292.7 + 13.33 \times 1913.6 + 28.704 \times 1534.4 + 84.536 \times 1155.3 + 191.701 \times 776.2 + 39.53 \times 397.1}{365.039} = 954 Da .$$

### Example 7

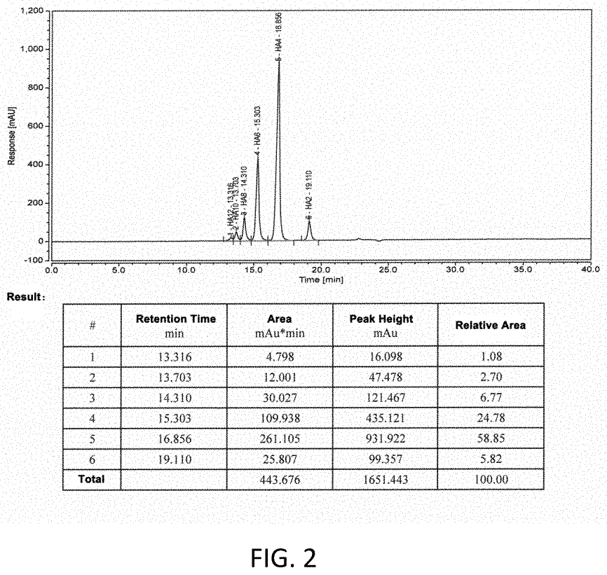

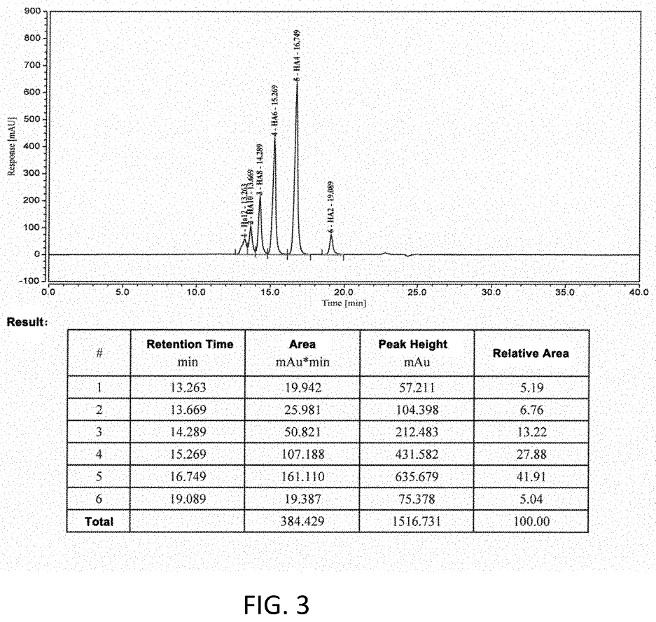

Another ultra-low molecular weight hyaluronic acid oligosaccharide mixture is obtained by using the enzymatic hydrolysis reaction solution of Example 2 according to the activated carbon adsorption process of Example 4 and the spray drying process of Example 6. Another ultra-low molecular weight hyaluronic acid oligosaccharide mixture is obtained by using the enzymatic hydrolysis solution of Example 3 according to the activated carbon adsorption process of Example 5 and the spray drying process of Example 6. FIG. 2 is a distribution spectrum of an ultra-low molecular weight hyaluronic acid oligosaccharide prepared in Example 2, and the molecular weight is calculated as 947 Da (calculated according to formula II). FIG. 3 is a distribution spectrum of an ultra-low molecular weight hyaluronic acid oligosaccharide prepared in Example 3, and the molecular weight is calculated as 1119 Da (calculated according to formula II).

### Example 8 of Structural Analysis

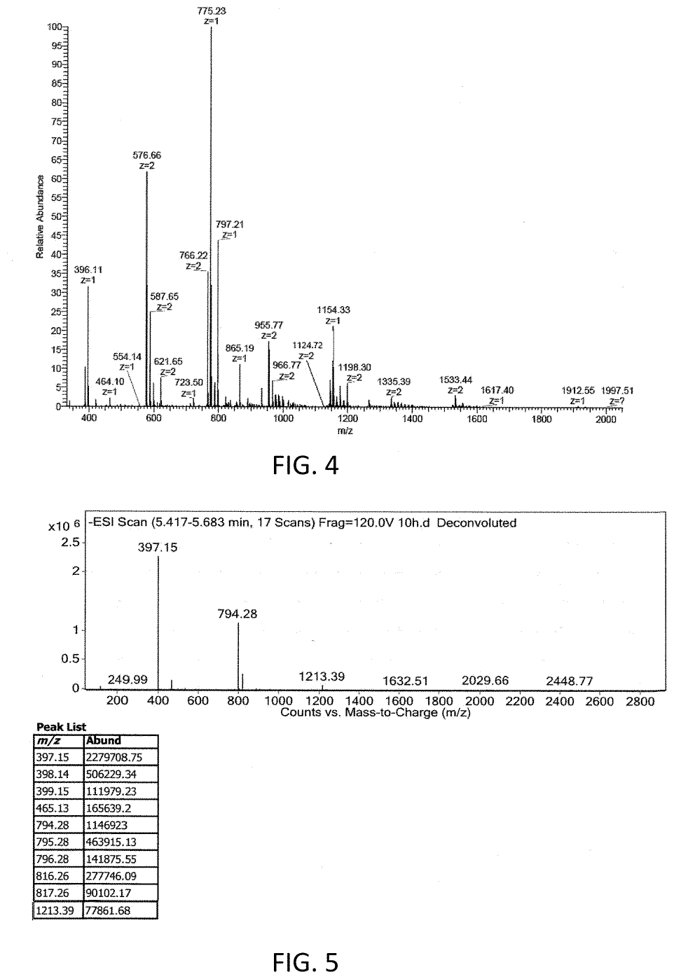

The molecular weight of the mixture of hyaluronic acid disaccharide to dodecaose obtained in Example 1 is measured by high-resolution mass spectrometry (HRMS), which is as illustrated in FIG. 4 and indicated in Table 1 specifically. Due to the partial or complete dissociation of the sodium ions of the hydrolyzing sodium hyaluronate in solution state, a plurality of charged ion peaks are present in the results of mass spectrometry.

The specific conditions of mass spectrometry are as follows:

Sheath gas flow rate: 40;
Aux gas flow rate: 10;
Sweep gas flow rate: 0;
Spray voltage: 3.5 kV;
Capillary temp.: 350;
S-Lens RF level: 55;
Aux gas heater temp.: 300;

### Polarity: Positive;

### Full MS Resolution: 70000;

Full MS Scan Range: 300 to 3000 m/z;

### Full MS Maximum IT: 200 ms;

AGC target 3e6.

TABLE 1
Molecular weight distribution of ultra-low molecular weight
hyaluronic acid oligosaccharides

| | | Theoretical molecular | m/z results for multiply |
|---|---|---|---|
| # | NAME | weight (Da) | charged ions |
| 1 | hyaluronic acid | 397.1 | [M − H] ⁻: 396.11 |
| | disaccharide (HA2) | | |
| 2 | hyaluronic acid | 776.2 | [M − 2H] ²⁻: 387.11 |
| | tetrasaccharide (HA4) | | [M − H] ⁻: 775.22 |
| | | | [M + Na − 2H] ⁻: 797.21 |
| 3 | hyaluronic acid | 1155.3 | [M − H] ⁻: 1154.33 |
| | hexasaccharide (HA6) | | [M − 2H] ²⁻: 576.66 |
| | | | [M + Na-3H] ²⁻: 587.65 |
| 4 | hyaluronic acid | 1534.4 | [M − H] ⁻: 1533.44 |
| | octasaccharide (HA8) | | [M − 2H] ²⁻: 766.22 |
| 5 | hyaluronic acid decaose | 1913.6 | [M − H] ⁻: 1912.55 |
| | (HA10) | | [M − 2H] ²⁻: 955.77 |
| | | | [M + Na − 3H] ²⁻: 966.77 |
| 6 | hyaluronic acid | 2292.7 | [M − 2H] ²⁻: 1145.33 |
| | dodecaose (HA12) | | |

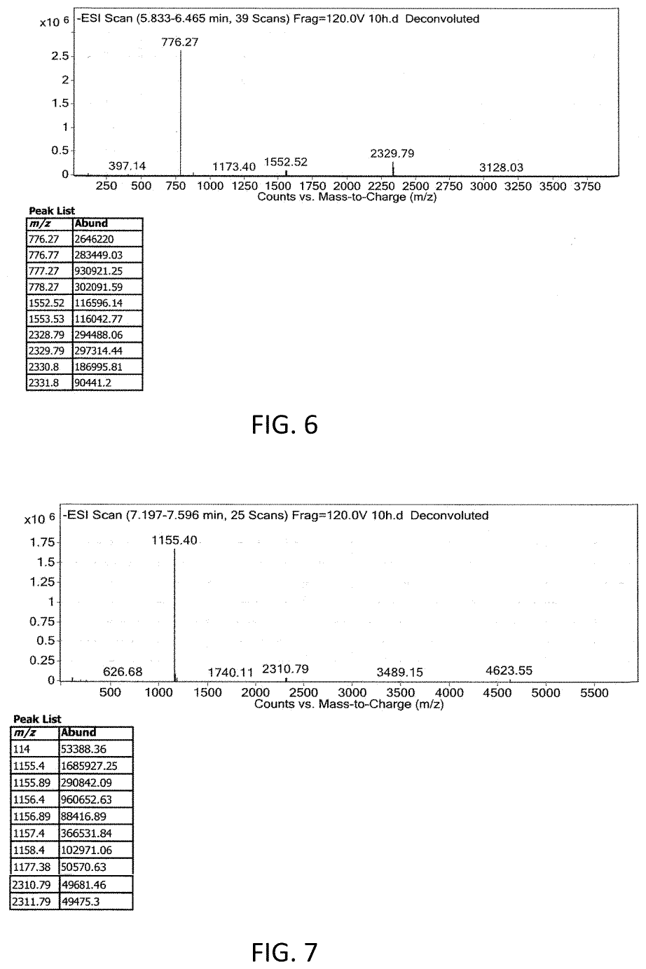

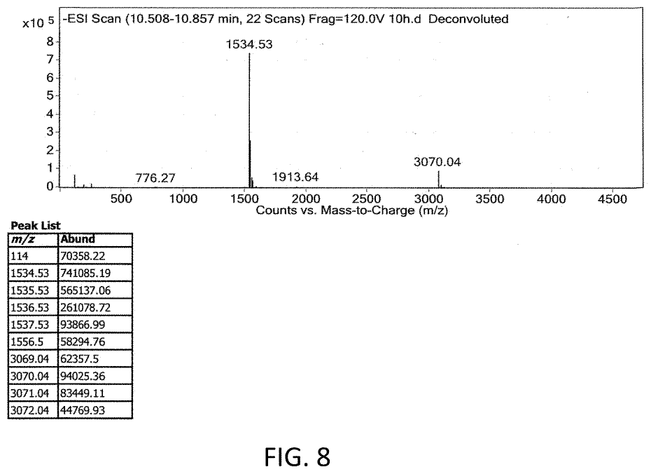

The mixture of hyaluronic acid disaccharide to dodecaose obtained in Example 1 is separated, and the mass spectrum of the obtained hyaluronic acid disaccharide is as illustrated in FIG. 5, and the mass spectrum of the hyaluronic acid tetrasaccharide is as illustrated in FIG. 6, and the mass spectrum of hyaluronic acid hexasaccharide is as illustrated in FIG. 7, the mass spectrum of hyaluronic acid octasaccharide and decaose is as illustrated in FIG. 8 (due to the relatively low content of decaose, the mass spectrum abundance is relatively low). The hyaluronic acid dodecaose is not detected because the content was too low.

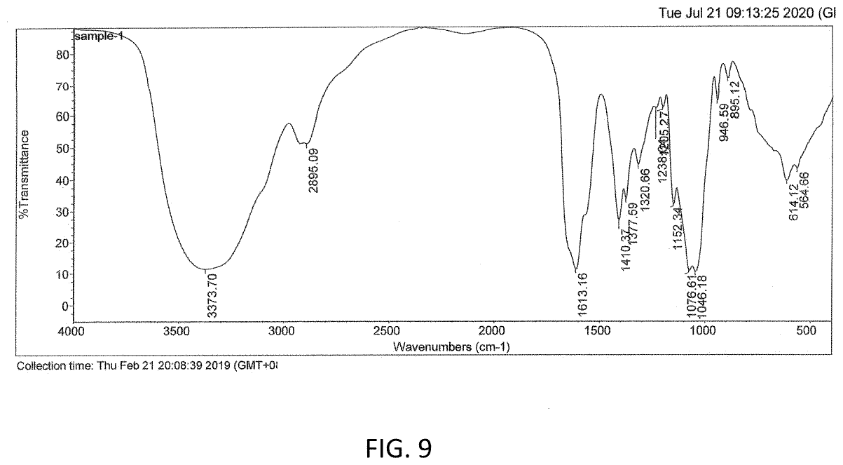

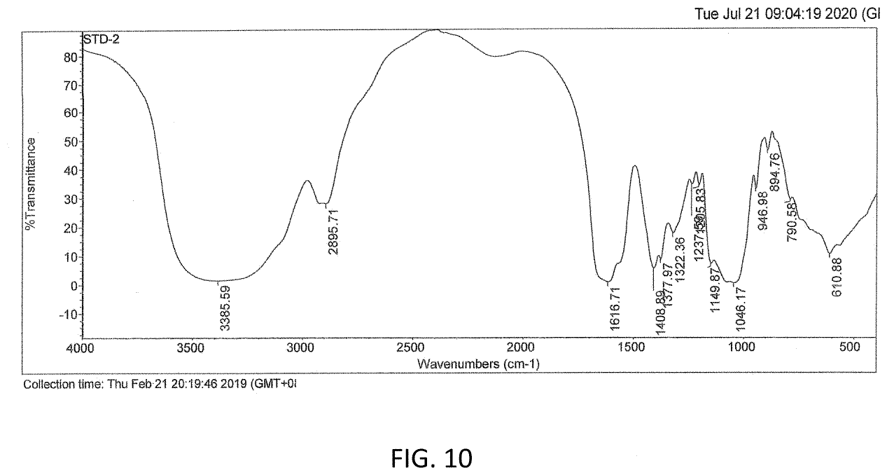

The infrared spectrum of the mixture of hyaluronic acid disaccharide to dodecaose obtained in Example 1 is measured as illustrated in FIG. 9. The results show that the infrared structure of the mixture of hyaluronic acid disaccharide to dodecaose obtained by the present disclosure is consistent with that of the macromolecular hyaluronic acid raw materials (FIG. 10), which proves that the unit structure of the mixture of hyaluronic acid disaccharide to dodecaose obtained by the present disclosure is not changed, and the specific infrared spectrum comparison is as indicated in Table 2 below.

TABLE 2
Infrared spectrum comparison of the mixture
of hyaluronic acid disaccharide to dodecaose obtained in the
present disclosure and macromolecular hyaluronic acid.

| | | wavenumber/cm⁻¹ | |
|---|---|---|---|
| | | macromolecule | Oligosaccharide |
| | | hyaluronic | composition of the present |
| # | Corresponding Group | acid | disclosure |
| 1 | VO—H OR VN—H | 3385.59 | 3373.70 |
| 2 | νC—H | 2895.71 | 2895.09 |
| 3 | νC═O | 1616.71 | 1613.16 |
| 4 | νC—N | 1408.89 | 1410.37 |
| 5 | νC—O | 1046.17 | 1046.18 |

### Example 9 for Detection of Efficacy and Activity

(1) Permeability and Hydration Properties

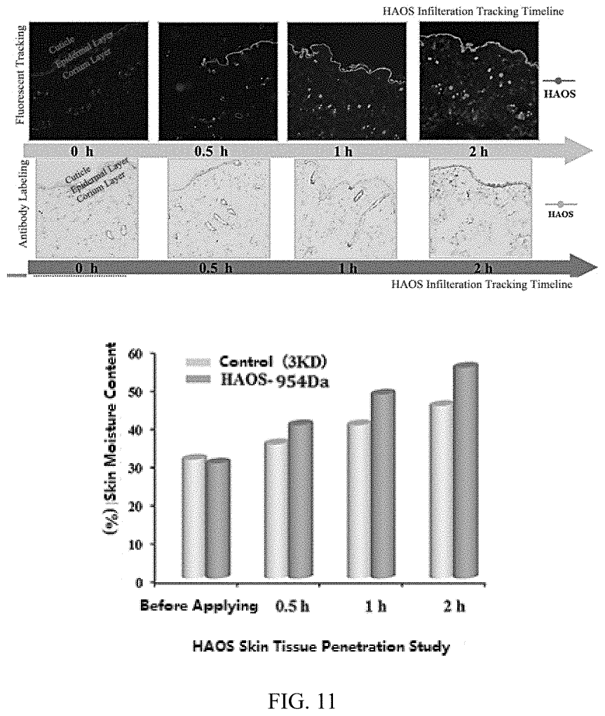

SD rat epidermis is taken as the experimental object, Hyaluronic Acid Binding Protein-Biotin bovine (Sigma, H9910) is used for immunohistochemistry or immunofluorescence, the permeability of the low molecular weight hyaluronic acid oligosaccharide mixture (HAOS) with a molecular weight of 954 Da obtained in Example 7 is investigated, after 0.5 h, 1 h and 2 h of application of HAOS of 954 Da and hyaluronic acid products with a molecular weight of 3 KDa in the market, the changes of epidermal skin moisture (MMV) of rats are measured to investigate the hydration characteristics, the results are as illustrated in FIG. 11, which shows that the HAOS with 954 Da molecular weight has a better permeation and moisturizing effect than 3 KDa molecular weight hyaluronic acid, the water content of rat skin with HAOS with 954 Da molecular weight is about 10% better than the commercially available 3 KDa molecular weight product after 5 mg/mL application for 2 hours.

(2) Restorative Effects on Human Immortalized Epidermal Cells

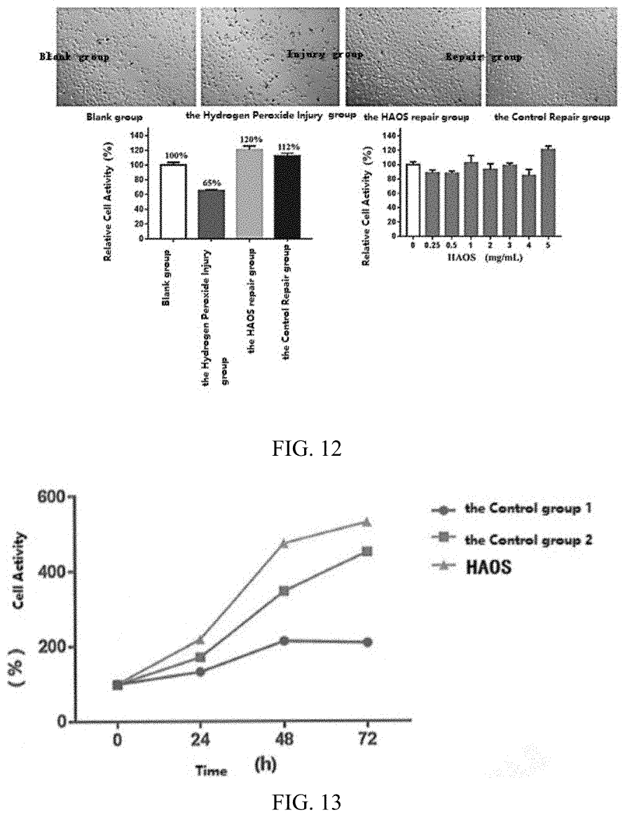

HaCaT cells (human immortalized epidermal cells) are taken as the experimental object, the cell viability is detected by CCK8, and the low molecular weight hyaluronic acid oligosaccharide mixture with a molecular weight of 954 Da obtained in Example 7 is investigated for its ability to promote the repair of cells damaged by hydrogen peroxide, the results are as illustrated in FIG. 12 that at the same concentration, the HAOS repair group has a higher relative cell activity of 120% compared with the blank group, the hydrogen peroxide injury group, and the control repair group (3 KD). The results show that the low molecular weight hyaluronic acid oligosaccharide mixture based on HAOS 954 Da has a better promoting repairing effect on the cells damaged by hydrogen peroxide at a concentration of 5 mg/mL compared with the commercially available 3 KDa molecular weight hyaluronic acid product. The repair rate is about 8% better than the commercially available 3 KDa molecular weight product.

(3) Repair Effect of Fibroblasts

A. Detection of the Cell Proliferation

Cell seeding: fibroblasts are seeded at a seeding density of 3.5E3 cells/well to a 96-well plate and incubated overnight in an incubator (37° C., 5% CO₂, 95% RH). The test is designed according to the following Table 3, and the specific detection results are as indicated in Table 4 and illustrated in FIG. 13 below.

The 954 Da hyaluronic acid used in the control group 1 is obtained by referring to the chemical cracking of macromolecular hyaluronic acid in CN101507733A, that is, 3 L of purified water and 330 g of macromolecular hyaluronic acid with a molecular weight of 800 KDa are add in a 5 L glass beaker, the stirring speed is controlled at 400 rpm, after all the substance are dissolved, the pH is adjusted to 2.5, and the 954 Da hyaluronic acid is obtained by hydrolysis at constant temperature 85° C. for 20 hours.

The 954 Da hyaluronic acid used in the control group 2 is obtained by referring to the chemical method in CN101507733A in combination with bovine testis-type hyaluronidase enzymatic hydrolysis of macromolecular hyaluronic acid, that is, 3 L of purified water and 330 g the macromolecular hyaluronic acid with a molecular weight of 800 KDa are added in a 5 L glass beaker, a stirring speed is controlled at 400 rpm, after all the substance are completely dissolved, the pH is adjusted to 2.5 and the 954 Da hyaluronic acid is hydrolyzed at a constant temperature of 85° C. for 12 h, then the temperature is lowered to 40° C., and 1.5×10⁸ U of bovine testis type hyaluronidase (CAS No.: 9001-54-1, commercially available) are added, the enzyme activity of the system is 5×10⁴ U/mL, the pH of the solution is adjusted to 5.5, and the system is kept at 40° C. and stirred for 12 hours.

TABLE 3
Design of Cell proliferation Test

| | | Culture | | | |
|---|---|---|---|---|---|
| Detection | | conditions | | | Detection |
| model | Group | (ingredients) | Dosage | Dosing time | Indicator |
| Fibroblasts | Control | 2% fetal | / | 0 h, 24 h, | Cell |
| | 1 | bovine serum + | | 48 h, 72 h | Proliferation |
| | | 954 Da | | | |
| | | hyaluronic | | | |
| | | acid obtained | | | |
| | | bypure | | | |
| | | chemical | | | |
| | | method | | | |
| | Control | 2% fetal | 2.5 mg/mL | | |
| | 2 | bovine serum + | | | |
| | | 954 Da | | | |
| | | hyaluronic | | | |
| | | acid obtained | | | |
| | | by chemical | | | |
| | | method and | | | |
| | | enzymatic | | | |
| | | hydrolysis of | | | |
| | | bovine | | | |
| | | testis-type | | | |
| | | hyaluronidase | | | |
| | HAOS | 2% fetal | 2.5 mg/mL | | |
| | of this | bovine serum + | | | |
| | invention | 954 Da | | | |
| | | HAOS | | | |
| | | obtained by | | | |
| | | enzymatic | | | |
| | | hydrolysis of | | | |
| | | leech-type | | | |
| | | hyaluronidase | | | |
| | | in Example 7 | | | |
| | | of the present | | | |
| | | invention | | | |

TABLE 4
Rsults of Cell Proliferation Assay

| | 0 h | | 24 h | | | 48 h | | | 72 h | | |
|---|---|---|---|---|---|---|---|---|---|---|---|
| | Relative | | Relative | | | Relative | | | Relative | | |
| | Vitality | SD | Vitality | SD | P | Vitality | SD | P | Vitality | SD | P |
| GROUP | % | % | % | % | value | % | % | value | % | % | value |
| Control 1 | 100% | 0.62 | 133 | 10.26 | / | 215 | 9.76 | / | 210 | 4.93 | / |
| Control 2 | 100% | 0.62 | 173 | 1.08 | 0.003 | 348 | 8.62 | 0.000 | 452 | 9.76 | 0.000 |
| HAOS of this invention | 100% | 0.62 | 220 | 0.62 | 0.000 | 475 | 4.35 | 0.000 | 531 | 8.62 | 0.000 |

The results shows that the cell viability of 954 Da HAOS obtained by enzymatic hydrolysis in Example 7 of the present disclosure at 2.5 mg/ml is significantly higher than that of control group 1 and control group 2 at 24 h, 48 h and 72 h (P<0.01). Compared with pure chemical cleavage or chemical method in combination with the commonly used bovine testis-type hyaluronidase method, the obtained 954 Da HAOS has a more obvious proliferating effect on fibroblasts.

B. Scratch Detection

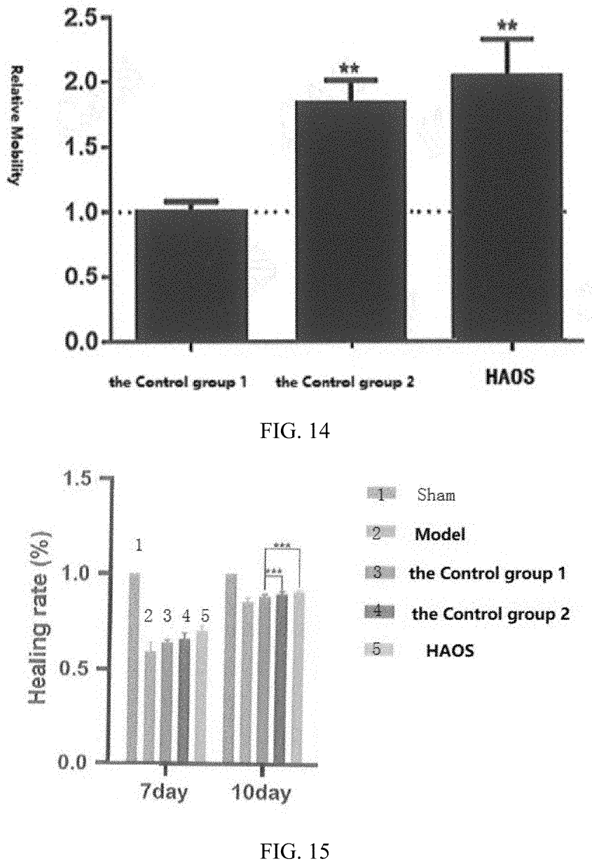

Cell seeding: fibroblasts are seeded at a seeding density of 2E5 cells/well to a 6-well plate and incubated overnight in an incubator (37° C., 5% CO₂, 95% RH). The test is designed according to Table 4 below, and the specific test results are indicated in Table 5 and illustrated in FIG. 14 below.

TABLE 4
Cell migration assay test

| Detection | Group | Culture Conditions | | Dosing | Detection |
|---|---|---|---|---|---|
| Model | | (ingredients) | Dosage | time | Indicator |
| Fibroblasts | Control 1 | 2% fetal bovine serum + | / | 24 h | Cell |
| | | 954 Da hyaluronic | | | Scratches |
| | | acid obtained by pure | | | |
| | | chemical method | | | |
| | Control 2 | 2% fetal bovine serum + | 2.5 mg/mL | | |
| | | 954 Da hyaluronic | | | |
| | | acid obtained by | | | |
| | | chemical method and | | | |
| | | enzymatic hydrolysis | | | |
| | | of bovine testis-type | | | |
| | | hyaluronidase | | | |
| | HAOS | 2% fetal bovine serum + | 2.5 mg/mL | | |
| | | 954 Da HAOS | | | |
| | | obtained by enzymatic | | | |
| | | hydrolysisof | | | |
| | | leech-type | | | |
| | | hyaluronidasein | | | |
| | | Example 7 of the | | | |
| | | present invention | | | |

TABLE 5
Results of Cell Migration Assay

| | Average | | Average of | Relative | |
|---|---|---|---|---|---|
| | of | Mobility | Relative | Mobility | P |
| Group | Mobility | SD | Mobility | SD | Value |
| Control 1 | 0.11 | 0.01 | 1.00 | 0.08 | / |
| Control 2 | 0.23 | 0.03 | 2.04 | 0.29 | 0.000 |
| HAOS of | 0.20 | 0.02 | 1.83 | 0.18 | 0.000 |
| This | | | | | |
| invention | | | | | |

The results show that the relative migration rate to fibroblasts of the 954 Da HAOS obtained by enzymatic hydrolysis in Example 7 of the present disclosure at 2.5 mg/ml was significantly higher than that of control group 1 and control group 2 (P<0.01). Compared with pure chemical cleavage or chemical in combination with the commonly used bovine testis-type hyaluronidase method, the obtained 954 Da HAOS has a more obvious effect on promoting the migration of fibroblasts.

(4) Effects on the Healing of Wounded Skin Tissue in Mice

Grouping: Sham group (sham operation group); model group (skin injury group, only application of distilled water); control group 1 (model+application of 954 Da hyaluronic acid obtained by pure chemical method); control group 2 (model+application of 954 Da hyaluronic acid obtained by chemical method and enzymatic hydrolysis of bovine testis-type hyaluronidase); HAOS of the present disclosure (model+application of 954 Da HAOS obtained by enzymatic hydrolysis of leech-type hyaluronidase in Example 7 of the present disclosure).

Except for the Sham group (sham operation group) that is not treated with skin injury, the other groups were conducted to create a 0.6 cm diameter round full-length skin excision open wound on the back of the mice to establish a model. The number of smearing is controlled for 3 times a day, 120 ul each time, and situations of the wound healing and scar formation are observed.

Through the measurement of the healing area and the calculation of the healing rate in the present disclosure, it is found that, on the 10th day of the injury, compared with control group 1 and control group 2, the healing rate for mouse skin damage is higher, and the healing of skin wounds can be significantly promoted by applying HAOS 954 Da obtained in Example 7 of the present disclosure.

1. An ultra-low molecular weight hyaluronic acid, characterized in that, an average molecular weight of the ultra-low molecular weight hyaluronic acid is less than 1200 Da, a distribution range of the molecular weight is narrow, the ultra-low molecular weight hyaluronic acid is a mixture of hyaluronic acid disaccharide to dodecaose; a content of hyaluronic acid disaccharide is 5-40%, a content of hyaluronic acid tetrasaccharide is 40-70%, a content of hyaluronic acid hexasaccharide is 10-30%, a content of hyaluronic acid octasaccharide is 1-15%, a content of hyaluronic acid decaose is 1-10%, and a content of that higher than hyaluronic acid decaose is less than 6%; a structural general formula of the ultra-low molecular weight hyaluronic acid is as shown in following formula I:

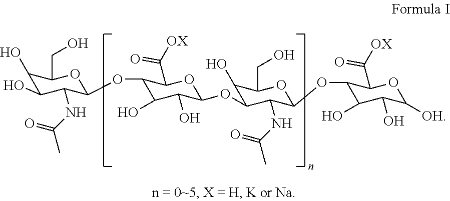

2. The ultra-low molecular weight hyaluronic acid according to claim 1, characterized in that the average molecular weight of the ultra-low molecular weight hyaluronic acid is 500-1200 Da, and further preferably 800-1000 Da.

3. The ultra-low molecular weight hyaluronic acid according to claim 1, characterized in that the ultra-low molecular weight hyaluronic acid is a mixture of hyaluronic acid disaccharide to dodecaose, the content of hyaluronic acid disaccharide is 5-10%, the content of hyaluronic acid tetrasaccharide is 50-70%, the content of hyaluronic acid hexasaccharide is 20-30%, the content of hyaluronic acid octasaccharide is 5-10%, the content of hyaluronic acid decaose is 1-5%, and the content of that higher than hyaluronic acid decaose is less than 3%.

4. A method for preparing the ultra-low molecular weight hyaluronic acid according to claim 1, characterized by enzymatically hydrolyzing a macromolecular hyaluronic acid raw material with hyaluronidase to obtain the ultra-low molecular weight hyaluronic acid with an average molecular weight of less than 1200 Da, wherein the distribution range of the molecular weight is narrow, the ultra-low molecular weight hyaluronic acid is a mixture of hyaluronic acid disaccharide to dodecaose; the content of hyaluronic acid disaccharide is 5-40%, the content of hyaluronic acid tetrasaccharide is 40-70%, the content of hyaluronic acid hexasaccharide is 10-30%, the content of hyaluronic acid octasaccharide is 1-15%, the content of hyaluronic acid decaose is 1-10%, and the content of that higher than hyaluronic acid decaose is less than 6%; a molecular weight of the macromolecular hyaluronic acid is equal to or greater than 1×10⁴ Da; the structural general formula of the ultra-low molecular weight hyaluronic acid is as shown in following formula I:

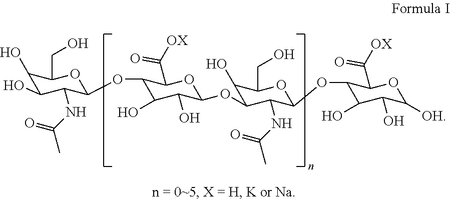

5. The method for preparing the ultra-low molecular weight hyaluronic acid according to claim 4, characterized in that the average molecular weight of the low molecular weight hyaluronic acid is 500-1200 Da, more preferably 800-1000 Da; the molecular weight of the macromolecular hyaluronic acid is equal to or greater than 1×10⁵ Da, more preferably 800 KDa-1600 KDa.

6. The method for preparing the ultra-low molecular weight hyaluronic acid according to claim 4, characterized in that the hyaluronic acid is a mixture of hyaluronic acid disaccharide to dodecaose, the content of hyaluronic acid disaccharide is 5-10%, the content of hyaluronic acid tetrasaccharide is 50-70%, the content of hyaluronic acid hexasaccharide is 20-30%, the content of hyaluronic acid octasaccharide is 5-10%, the content of hyaluronic acid decaose is 1-5%, and the content of that higher than hyaluronic acid decaose is less than 3%.

7. The method for preparing the ultra-low molecular weight hyaluronic acid according to claim 4, characterized in that the hyaluronidase is a leech-type hyaluronidase, which is obtained by optimized expression of yeast.

8. The method for preparing the ultra-low molecular weight hyaluronic acid according to claim 4, characterized in that operating conditions of the enzymatic hydrolysis reaction are as follows: an addition amount of the hyaluronidase relative to a reaction solution is 1×10⁴ U/mL to 1×10⁵ U/mL, a concentration of the macromolecular hyaluronic acid raw material is 40-200 g/L, and a reaction solvent is purified water, an enzymolysis time is 12-36 hours, an enzymolysis temperature is 35-45° C., a stirring speed is 100-700 rpm, and an enzymolysis pH is 4.0-6.0.

9. The method for preparing the ultra-low molecular weight hyaluronic acid according to claim 8, characterized by heating the reaction solution to 80-90° C. after the enzymolysis reaction, keeping the temperature for 30-60 minutes for inactivation, cooling the temperature to below 50° C., adding activated carbons for adsorption, and then filtering and sterilizing the reaction solution by a 0.22 μm capsule-type filter element for a spray-drying.

10. The ultra-low molecular weight hyaluronic acid according to claim 1, characterized in that, compared with an ordinary low molecular weight hyaluronic acid, the obtained ultra-low molecular weight hyaluronic acid has better skin permeability and property for promoting a damaged skin to be repaired and has more obvious effect of promoting the repair of human immortalized epidermal cells damaged by hydrogen peroxide.

11. The ultra-low molecular weight hyaluronic acid according to claim 1, characterized in that, the obtained ultra-low molecular weight hyaluronic acid has more obvious effects of promoting proliferation and migration on fibroblasts than the hyaluronic acid oligosaccharide obtained by pure chemical cleavage or chemical method in combination with the bovine testis-type hyaluronidase enzymolysis.

12. The ultra-low molecular weight hyaluronic acid according to claim 1, characterized in that, the obtained ultra-low molecular weight hyaluronic acid, has a higher healing rate on mouse skin injury and can significantly promote the healing of skin wounds, compared with hyaluronic acid oligosaccharide obtained by pure chemical cleavage or chemical method in combination with the bovine testis-type hyaluronidase enzymolysis.

13. The ultra-low molecular weight hyaluronic acid according claim 1, characterized by a use of the ultra-low molecular weight hyaluronic acid in the fields of preparing medicines, cosmetics and health care products.
